(19) United States
(12) Patent Application Publication
(10) Pub. No.: US 2025/0058073 A1
(43) Pub. Date: Feb. 20, 2025

(54) DEVICE AND METHOD FOR USE IN TREATMENT OF PATHOGENS

(71) Applicant: KAMAGTECH BIO PHARMA, Or Akiva (IL)

(72) Inventor: Shlomo Uri HAIMI, Or Akiva (IL)

(21) Appl. No.: 18/721,676

(22) PCT Filed: Dec. 19, 2021
(86) PCT No.: PCT/IB2021/061996

§ 371 Date: Jun. 19, 2024

(51) Int. Cl. A61M 16/10; A61M 16/00; A61M 16/12; A61M 16/14; A61M 16/20

(52) U.S. Cl. CPC A61M 16/108; A61M 16/0063; A61M 16/12; A61M 16/14; A61M 16/201; A61M 2202/0007; A61M 2202/02; A61M 2202/0468; A61M 2205/0205; A61M 2205/07; A61M 2205/3368; A61M 2205/36

(57) ABSTRACT

A method and device for providing an inhalable heated dry gas to a user, the device including: an air pump adapted to draw ambient dry gas into the device and pump the gas through the device; a heating component adapted to beat the dry gas pumped by the air pump, resulting in dry, heated gas; and an outlet port through which the heated dry gas is expelled from the device.

### FIELD OF THE INVENTION

The present invention relates to a healthcare apparatus for application towards respiratory systems of living creatures. More specifically, the present invention relates to a medical device for treatment and prevention of infections caused by pathogens that enter the body of humans and animals via the respiratory tract.

### BACKGROUND OF THE INVENTION

Studies show that pathogens, including viruses, bacteria, and fungi enter the body in a variety of ways. A common way for pathogens to enter the body is via the upper respiratory tract, including airborne and droplet transmission.

Pathogenesis generally involves an incubation period which is defined as the time between exposure and the onset of symptoms. For respiratory tract-based transmission this includes the period of pathogen entry into the body, reaching the target host niche and replication. The period in which the pathogen has not yet attached to and/or entered a cell, or even traveled deeper into body tissue-is a window of opportunity for interference and disease intervention.

Relative lower temperatures, relative low humidity levels, unionized environments and, in some cases, cytokine absent environments support prolonged survival of pathogens on contaminated surfaces, including those pertaining to the respiratory system.

### SUMMARY OF THE INVENTION

Each of the at least one of: high relative temperatures, high relative humidity levels, certain ionization degrees, ultraviolet radiation and cytokine enriched environments have substantial effect on inactivation of pathogens.

Arguably the combination of the at least two of: high relative temperatures, high relative humidity levels, certain ionization degrees ultraviolet radiation and cytokine enriched environments has a synergetic effect on inactivation of pathogens.

The inactivation of pathogens is demonstrated by eradication of pathogens while still in the upper respiratory tract. Delayed activation of pathogens is demonstrated by creation of unfavorable conditions for pathogens to attach to and or enter a cell or travel deeper into body tissue, thus assisting the host immune system to fight those pathogens and prevent pathogen driven disease.

According to the present invention there is provided a device for providing an inhalable heated dry gas to a user, the device including: an air pump adapted to draw ambient dry gas into the device and pump the gas through the device; a heating component adapted to heat the dry gas pumped by the air pump, resulting in dry, heated gas; and an outlet port through which the heated dry gas is expelled from the device.

According to further features in preferred embodiments of the invention described below, the air pump is an air compressor, wherein the pumped dry gas is compressed by the air compressor.

According to still further features in the described preferred embodiments the heating component is selected from the group consisting: a heating element, a laser, an infrared light.

According to still further features the device further includes an electrical source, the electrical source adapted to ionized the heated gas. According to still further features the dry gas is heated to a temperature in a range between 40° C. and 70° C. According to still further features the dry gas is heated to a temperature in a range between 50° C. and 60° C.

According to still further features the dry gas is pumped through a pipe disposed within the device. According to still further features the outlet port further includes an ultraviolet light source.

According to still further features the device further includes a gas inlet port adapted to communicate therethrough an added gas from an external source to intermix with the dry gas. According to still further features the added gas is an ionized gas. According to still further features the inlet port further includes a valve adapted to selectively control access of the added gas into the device so as to intermix with the dry gas.

According to still further features the device further includes a reservoir for holding a disinfectant additive to be intermixed with the dry gas in the device. According to still further features the device further includes a cytokine compartment for holding a cytokine additive to be intermixed with the dry gas in the device.

According to another embodiment there is provided a method of providing a heated dry gas, the method including: pumping a dry gas into a device and propelling the dry gas through an internal volume of the device with an air pump of the device; heating the dry gas inside the device by a heating component of the device so as to receive a heated dry gas; and expelling the heated dry gas out of an outlet port of the device.

According to further features the method further includes the step of compressing the dry gas with a compressor. According to further features the method further includes the step of ionizing the dry gas.

According to further features the method further includes the step of adding a disinfectant fluid to intermix with the dry gas. According to further features the method further includes the step of adding a cytokine fluid to intermix with the dry gas.

According to further features the method further includes the step of adding an added gas to intermix with the dry gas. According to further features the added gas is an ionized gas. According to further features the method further includes the step of radiating the dry gas with ultraviolet radiation.

### BRIEF DESCRIPTION OF THE DRAWINGS

Various embodiments are herein described, by way of example only, with reference to the accompanying drawings, wherein:

FIG. 1 is a schematic diagram of a device according to the immediate invention;

FIG. 1A is a schematic diagram of a device 10′ according to another embodiment of the immediate invention;

FIG. 2 is a flow diagram of a process 200 for preparing and using device 10.

### DESCRIPTION OF THE PREFERRED EMBODIMENTS

The principles and operation of a medical device according to the present invention may be better understood with reference to the drawings and the accompanying description.

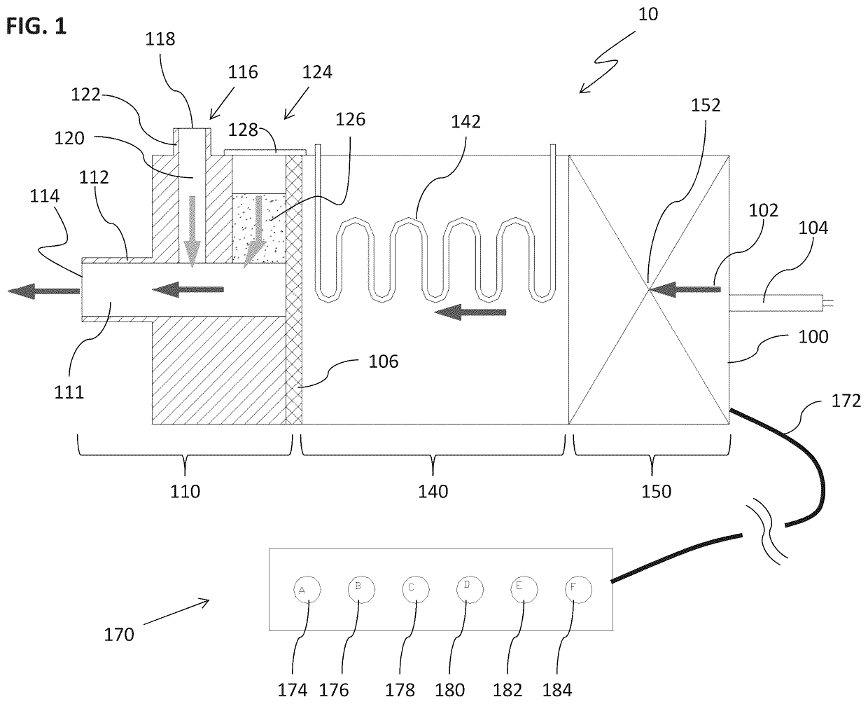

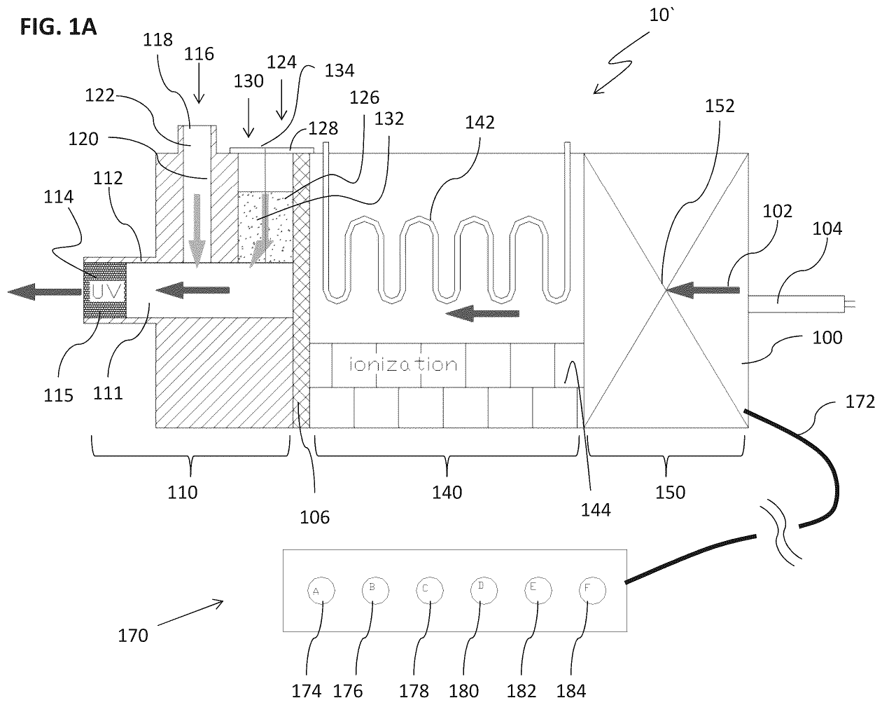

Referring now to the drawings, FIG. 1 illustrates a schematic diagram of a device according to the immediate invention. The device may be a personal device, i.e. adapted for home use; or it may be an institutional device adapted for multiple users. The multiple users may be multiple simultaneous users or simply multiple users, one after the other (sequential users). The device 10 is adapted to output an inhalable heated dry gas that can be used in the treatment and prevention of infections caused by pathogens that enter the body of humans and animals via the respiratory tract.

FIG. 1 refers to a device 10 for use by a single user (at any given time). The device has three main sections, preferably housed in a single housing 100. According to other embodiments, the sections may be housed in one or more separate (but connected) housings (either each section housed separately, or two sections housed together, and a third section housed separately). As noted, in embodiments with separate housings, the housings/sections are in fluid communication with each other. The direction of the fluid communication is indicated in FIG. 1 by arrows 102.

For the purposes of the description of the invention, three separate section are depicted and described. However, it is made clear that two or more of the sections may be amalgamated into a single section. Alternatively, or additionally, the device may be subdivided into more than three sections. In summary, the division of the apparatus into sections is not intended to be limiting in any way be merely employed to ease reading and clarify understanding of the subject matter.

A first section 110 contains the user interface components: e.g. an outlet port having an ultraviolet lamp (to which a mask and flexible tubing can be attached), fluid passage, added ionized gas/oxygen inlet port and disinfectant reservoir. A second section 140 (moving further away from the user) includes the heating apparatus (e.g. heating element, infrared light source, laser source etc.). A third section 150 includes the compressor/air pump etc. which provides the flow of air for the apparatus.

The device 10 is now described according to the direction of use, i.e. from initial air intake to output through the user interface components. In the third section 150, a pump 152 sucks air from the area outside the housing of the device. In preferred embodiments, the pump is an air compressor (not shown) that both compresses and pumps the air in the indicated direction. In other embodiments, there is no compressor. The air (compressed or not compressed) is propelled through the apparatus in the direction of arrows 102.

Air enters the second section 140 from the third section 150. The air may be conveyed through an opening/passage (not shown) between the sections 150, 140. Alternatively, or additionally, the air may be conveyed between the two section through a conduit (not shown), e.g. when the sections are housed separately.

A heating component heats the air in the second section. The heating component may be a heating element 142 (as depicted) or some other manner of heating such as a laser or infrared lamp. The type of heating component is not intended to be limiting, such that any mechanism or method of heating the dry gas within the instant apparatus, that would be obvious to one skilled in the art after being made aware of the invention, is considered to be within the scope of the invention. Preferably, the dry gas is heated to ˜40-70° C. in order to effectively denature and inactivate the pathogen. More preferably, the dry gas is heated to ˜50-60° C.

The second section 140 may be a compartment holding both the air and the heating component (as depicted). Alternatively, the air may be conveyed inside a pipe, tubing, or the like (not shown) while the heating component is outside the pipe/tubing, such that the second section includes both the heating component and the piping/tubing.

The first section 110 is a user interface compartment. The user interface compartment includes an outlet port 112 through which the heated dry gas (e.g. air) is expelled out of the apparatus 10. In some embodiments (see FIG. 1A), the heated dry gas is also ionized. The heated air is communicated through a fluid passage 111. The passage 111 terminates at an egress opening 114. Part of the terminal end of the passage is surrounded by outlet port 112. Flexible tubing and a face mask can be attached to outlet port 112 as is known in the art. In some embodiments (see FIG. 1A), an ultraviolet lamp 115 is provided at either the egress opening 114 or the face mask. In some embodiments, the outlet port includes a pressure regulator (not shown) for regulating the speed at which the heated dry gas exits the apparatus. Alternatively, the pressure regulator may be part of the mask and tubing, attached to the outlet.

In preferred embodiments, as depicted in the Figure, device 10 further includes a gas inlet port 116. The gas inlet port 116 provides an ingress and/or fluid conduit through which oxygen or a different gas or a composition of gases can be added to, and mixed with, the heated air in fluid passage 111, prior to the same exiting through opening 114. In an alternative option, the added gas is an ionized gas. The inlet port 116 is depicted in the Figure as being located in the first section 110, in fluid communication with fluid passage 111. The foregoing (and following) detailed description is provided in relation to this depiction. The aforementioned notwithstanding, it is made clear that the inlet port may alternatively be located in one of the other sections of the device, such that the position or location of the inlet port is not considered limiting in any way.

For added clarity, the inlet port 116 is defined as being a fluid conduit in potential fluid communication with the internal volume of device 10. The internal volume of device 10 is adapted for gaseous contents to be communicated there-through, entering at the air pump 152 and exiting at the egress opening 114. The inlet port 116 is adapted to facilitate communication of an added gas from an external source into the internal volume of device 10 (e.g. into fluid passage 111; and preferably through an airtight, unidirectional coupling) in order to intermix with the gaseous contents of device 10.

The inlet port preferably includes connectors, fasteners, valves, gaskets, etc. to ensure an airtight connection to the outlet port. For example, the inlet port may include a unidirectional valve adapted for receiving a hose from a gas container; the inlet port mouth including a gasket for creating an airtight seal with the hose when inserted therein; the hose or the pressured gas communicated there-through traversing the unidirectional valve without allowing the heated air to escape. In some embodiments, the air in device 10 will be under positive pressure (e.g. to prevent contaminants from entering the device). In such cases, the pressure under which the added gas is introduced through the inlet port must be higher than the positive pressure of the contents of the device. Introduction of the added gas can be controlled from the external source or from the device 10. The unidirectional valve may be a mechanical valve which is breached under pressure or by penetration of the tubing/piping. For example, the valve may be a normally-closed plastic flap or a spring loaded plastic flap; in either example, the valve is held closed until either pushed open by gas under pressure or penetrated by a hose or tubing or the like. Alternatively, the valve may be electromechanical, such as a solenoid valve, whose operation is actuated and controlled by controls on an external panel.

For added clarification, it is noted that the inlet port includes a mouth or opening 118, a fluid conduit 120 and a connector port 122. The external added gas source can be a gas cylinder (e.g. an oxygen tank) or a built-in gas connection (e.g. an oxygen wall connection in a hospital) that is connected to the inlet port in any manner. Usually, the connection is via a flexible tube or pipe. Alternatively, a gas canister can be coupled directly to the inlet port, by mechanically coupling the outlet of the canister to the inlet port. The coupling can be performed by either placing the tubing or mechanical connector over the connector port 122 or inserting it into mouth 118. If needed, a gasket/O-ring can be employed to ensure a hermetic connection. The coupling connection can also be a twist-lock connection, as is known in the art.

Preferably the inlet port includes a valve (not shown but discussed above). The valve may be a simple mechanical valve (e.g. located near the mouth of the inlet port) or an electromechanical valve (e.g. located between the distal end of the fluid conduit and the fluid passage). The valve adapted to selectively control access of the added gas into the device so as to intermix with the dry gas (prior to the gas being heated, during the heating process or after the dry gas has been heated).

In preferred embodiments (such as depicted in FIG. 1), device 10 further includes a reservoir 124 for holding a disinfectant additive to be intermixed with the dry gas in the device (prior to the dry gas being heated, during the heating process or after the dry gas has been heated). The term disinfectant is used herein as a hold-all term for an antiviral, antibacterial, antimicrobial, antifungal and antiseptic. It is not the intention that the term refer to all of the above but rather to at least one of the above.

The precise composition of a disinfectant for use in conjunction with the instant device 10 is not necessarily an object of the instant invention. Nonetheless, for the sake of completion, some relevant details with regard thereto are discussed hereafter. At present, there is a worldwide coronavirus pandemic. Coronavirus disease 2019 (COVID-19) is a respiratory infection caused by SARS-CoV-2 (COVID-19 virus). The COVID-19 virus is transmitted mainly through close physical contact and respiratory droplets.

Like other coronaviruses, SARS-CoV-2 is an enveloped virus with a fragile outer lipid envelope that makes it more susceptible to disinfectants compared to non-enveloped viruses such as rotavirus, norovirus and poliovirus.

The World Health Organization (WHO) has published an interim guidance regarding cleaning and disinfection of environmental surfaces in the context of COVID-19. While the prescribed disinfectant composition is not necessarily applicable, it is somewhat instructive. Medical professionals and regulatory bodies will formulate and approve one or more compositions of non-toxic, inhalable disinfectants for use with the instant innovative device.

The interim guidance advises the following composition for the disinfection of surfaces in healthcare settings: (a) Ethanol 70-90%; (b) Chlorine-based products (e.g., hypochlorite) at 0.1% (1000 ppm) for general environmental disinfection or 0.5% (5000 ppm) for blood and body fluids large spills; and (c) Hydrogen peroxide >0.5%.

It is clear that chlorine-based products are not appropriate for internal use (i.e. inhaling), leaving ethanol and hydrogen peroxide in the prescribed concentrations. Besides for ethanol/alcohol and hydrogen peroxide, various other non-toxic disinfectants may alternatively or additionally be used. Both vinegar and essential oils are cited as non-chemical alternatives that have disinfecting characteristics. Lavender and tea tree essential oils are listed as having antibacterial (at least) effect. Other essential oils including geranium, lemon, orange, eucalyptus, rosemary, cinnamon, clove, thyme and peppermint are reported to have antiseptic, antiviral, antimicrobial, antifungal and/or antibacterial properties.

Dry gas of the instant innovative devices and methods is preferable to a moist gas for the intended purposes of the innovation. Moist air that remains in the body's tubes and canals is a fertile ground for the proliferation of bacteria, viruses and the like. Hot air is effective in the respiratory tract and causes the destruction of pathogens (at levels of 50-60 degrees Celsius)¹. ^{1 “}The Effects of Temperature and Relative Humidity on the Viability of the SARS Coronavirus” Chan et al. Hindawi Publishing Corporation, Advances in Virology, Volume 2011, Article ID 734690

There is provided herein a therapy for treating patients with Covid-19. Providing hot, dry air is the initial stage in the procedure for providing care. Experiments were performed with the addition of medical ethanol at levels of 40% and found that the spray significantly increases and enhances the healing effect.

Experiments done in human trials have shown great relief for Covid-19 patients, as well as patients suffering from throat and lung infections. Further experiments will be performed in the future. These are the steps in the treatment process:
1. Dry hot air.
2. Ethanol spray 40% or more.
3. Combination of controlled ionization (with hot air and with ethanol liquid spray).
4. Oxygen if necessary
5. Medication bronchodilators (if necessary)
6. All stages are synchronized for breathing i.e., when a person inhales air in [normal breathing] the system puts in hot air (with or without liquid spray). When the person exhales the system pauses and the air—after affecting the infected areas—is easily expelled from the body.

It is noted that the basis of the treatment is hot, dry air. Even if a liquid disinfectant is added, this does not detract from the basic feature of hot, dry air that is not based on a liquid. This is antithetical to the use of moist gasses, even those liquid solutions that are atomized, vaporized and/or aerosolized. Even the added liquid disinfectant is antithetical to the methods that use liquid solutions, as the “liquid” particles added to the hot dry air likewise serve to destroy the bacteria and viruses without providing a climate for propagation of the bacteria and viruses etc. The instant methods and devices fly in the face of the accepted practice for the last 50 years of using hot (or cold) humidifiers for treating respiratory distress.

In all the devices and processes detailed herein, if necessary, the ambient air that is pumped into the device may be de-humidified to ensure that the gas inside the device is dry gas.

FIG. 1A is a schematic diagram of a device 10′ according to another embodiment of the immediate invention. Device 10′ has the same components, specifications and functionality as device 10 of FIG. 1, with certain additional components. It is made clear that the entire description applies equally to both FIGS. 1 and 1A and to devices 10 and 10′ except for specific descriptions of components that appear in one of the devices but not in the other and vice-versa. Generally, such specific descriptions will be prefaced with wording such as “in some embodiments”, “in another embodiment” or “in the embodiment of FIG. 1A” and the like. Again, any description that can be applied to both figures and devices is to be seen as if fully set forth for each device, mutatis mutandis.

In FIG. 1A device 10′ further includes a cytokine compartment 130 for holding a cytokine additive to be intermixed with the heated dry gas in the device. The term “cytokine” is used herein to include immune cells like macrophages, B lymphocytes, T lymphocytes and mast cells, as well as endothelial cells, fibroblasts, and various stromal cells. It is not the intention that the term refer to all of the above but rather to at least one of the above.

FIG. 1A further includes a component for ionizing the dry gas in the heating chamber 140. It is noted that at sufficient level of heat, e.g. from the heating element, the gas can become ionized. Nonetheless, chamber 140 further includes an electrical source, such as a transformer 144. The electrical source is adapted to ionize the gas inside the device 10′ (the ionization process may take place before heating the gas, during the heating process or once the gas has already been heated).

The following description applies to both FIGS. 1 and 1A. Referring now to the structural aspects of the reservoir 124, the reservoir includes a container 126 and a cover 128. In the embodiment of FIG. 1A, the reservoir further includes the cytokine compartment 130 which is made up of a container 132 and cover 134. Of course, in other configurations, a single lid can cover both containers.

In preferred embodiments, a unidirectional valve (not shown) is interposed between the container 126 and the fluid passage 111. Exemplarily, another unidirectional valve can be located between compartment 132 and fluid passage 111. Alternative configurations can include a common area within reservoir 124 where the cytokine additive mixes with disinfectant prior to being added to the fluid passage.

In embodiments, the valve(s) is a mechanical valve, manually operated. In other embodiments, the valve(s) is an electromechanical valve, such as a solenoid. The solenoid can be electrically actuated via an external control panel or even a remote control. Alternatively, the canister(s) can be a vacuum stopped container, like a syringe, having a small opening on one end and a plunger on the other end. The plunger(s) may be a mechanical plunger which is actuated manually or an electromechanical plunger which operation is automated and actuated/controlled by a connected control panel or wireless remote.

During operation (discussed in further detail below), an amount of disinfectant and/or cytokines can be introduced into the heated dry gas, whether ionized or not, inside the fluid passage. As mentioned, the introduction of the disinfectant can be performed manually or automatically. Preferably, the device includes precise controls (manual or automatic) for controlling the amount of disinfectant introduced into the heated dry gas, whether ionized or not. The disinfectant and/or cytokine additive may be introduced alone or in addition to the added gas, whether ionized or not. Alternatively, the added gas may be introduced to the heated gas in the inner volume of the container, without a disinfectant or cytokines being added. In still another alternative, the heated dry gas (air) may be used without the added gas, the disinfectant, or the cytokines.

The disinfectant may be provided in the container in a liquid or a gas state. The reservoir is adapted to release the disinfectant as a liquid or a gas. The container may be, for example, a replaceable pressurized canister, adapted to release the disinfectant as a gas. Alternatively, the canister may be a fixed container (possibly removable for cleaning purposes, but otherwise not intended to being removed) into which liquid disinfectant is poured. The disinfectant is released or expelled from the container in a gas or liquid form, as mentioned. If in a liquid form, the pressurized gas in the internal volume of the device (e.g. in the fluid passage) will aerosolize the liquid converting it into a gas state for output via the outlet port. In preferred embodiments, the cytokine additive is provided in a similar fashion to the disinfectant, i.e. with similar alternatives and options.

As with the inlet port 116, it is made clear that the reservoir 124 and/or compartment 130 may alternatively be located in one of the other sections of the device, such that the position or location of the reservoir 124 or compartment 130 is not considered limiting in any way. Compartment 130 may be a completely distinct container placed side-by-side or in separate locations.

For added clarity, the reservoir 124 is defined as being a fluid container in potential fluid communication with the internal volume of device 10. The reservoir 124 is adapted to facilitate communication of a disinfectant [fluid] from the container into the internal volume of device 10 (e.g. into fluid passage 111), via a mechanical or electromechanical valve, in order to intermix with the gaseous contents of device 10.

For added clarity, the compartment 130 is defined as being a fluid container in potential fluid communication with the internal volume of device 10′. The compartment 130 is adapted to facilitate communication of a cytokine [fluid] from the container into the internal volume of device 10′ (e.g. into fluid passage 111), via a mechanical or electromechanical valve, in order to intermix with the gaseous contents of device 10°.

According to one embodiment, as depicted in FIG. 1, device 10 is powered by power mains via an electrical cable 104. In another embodiment, device 10 is battery powered, e.g. by a rechargeable battery. The rechargeable battery may be rechargeable in situ, by coupling the battery to power mains, e.g. via cable 104. Alternatively, the battery may be removable and/or rechargeable, e.g. at a charging station. In yet another embodiment, device 10 may be powered via mains or by a battery.

Preferably, as depicted in FIG. 1, a filter 106 interposes the second section and the first section. Alternatively, or additionally, the filter may interpose between the second section and the third section.

Optionally, the mask has an arrangement whereby excess air not inhaled by the user is returned to the device, preferably through a filter, e.g. via a high-efficiency particulate air (HEPA) filter. Optionally, where not provided on the outlet port, the mask also includes an ultraviolet lamp for ultraviolet radiation.

A control panel 170 is in electronic communication with device 10. The control panel may be embodied on the housing of the device, or detachable but connect to the housing or even a remote-control panel in wireless electronic communication with device 10. In the exemplary embodiment depicted in FIG. 1, the control panel is in wired communication with the housing of device 10 via cable 172.

Dial A 174 is a temperature control. In some embodiments device 10 (e.g. on panel 170 or elsewhere on the housing 100) further includes a temperature gauge (not shown) indicated the temperature of the heated dry gas in the device 10.

Dial B 176 control the speed at which the heated dry gas is released from the device. There are at least two options for releasing the gas into the tubing and mask, slow or fast: (1) a relatively slow release of a predetermined amount of the heated air (with or without disinfectant and/or added gas and/or cytokines and/or ultraviolet radiation); and (2) a relatively fast release or one-time release, where a predetermined amount of heated dry gas (with or without disinfectant and/or added gas and/or cytokines and/or ultraviolet radiation) is released all at once, or very quickly. According to embodiments, the same amount of heated gas is released, regardless of the method employed, the difference being the amount of time over which the gas is release. Alternatively, the first method does not release a predetermined amount of gas but rather the gas is released for a predetermined amount of time, or for an undetermined amount of time, being switched off manually by an operator. The pressure under which the heated dry gas is outputted to the patient is regulated to ensure pressurized gas does not push pathogens from the respiratory tract to the lungs.

A dial C 178 operates to select or regulate the flow rate of the disinfectant fluid (gas or liquid). According some embodiments and configurations the heated air is stored under pressure prior to release/use, and only once the heated gas is being released is the disinfectant also released into the gaseous mix. In such embodiments or configurations, dial C controls the flow rate of the disinfectant into the pressurized, heated air in fluid passage 111, thereby effecting the desired ratio of disinfectant to air/gas. Of course, the rate of release of the heated gas (controlled by dial B) is another variable that contributes to the aforementioned ratio.

A dial D 180 regulates the flow rate of the added gas which is communicated from the external source via inlet port 116. The added gas flow rate is the third variable that dictates the ratio between the native, pressurized, heated gas, and the disinfectant and the added gas. If cytokines are added, the cytokines introduce yet another variable into the composition of the fluid within of the device. As mentioned already numerous times, one or more of the added gas, the disinfectant, the cytokines and the ultraviolet radiation may not be included in the released gas.

A button E 182 is a notification or alert button. For example, if there is a malfunction, the button flashes red or otherwise alerts the operator and advises the operator to press the button to stop action of the device or otherwise solve the compromised situation.

A button F 184 is an activation/release button which activates the heated gas release mechanism. Alternatively, button F 184 may function to regulate the release of the cytokine additive of device 10′. For device 10′, button F 184 operates to select or regulate the flow rate of the cytokine fluid (gas or liquid). In the latter case, an additional user control, not shown, is actuated to activate (and deactivate) the release mechanism. According to some embodiments and configurations the heated air is stored under pressure prior to release/use, and only once the heated gas is being released is the cytokine additive also released into the gaseous mix. In such embodiments or configurations, dial F controls the flow rate of the cytokine additive into the pressurized, heated air in fluid passage 111, thereby effecting the desired ratio of cytokine to air/gas.

The direct heat eradicates (usually at ˜50° C.) most of the pathogens, while having a prolonged negative effect on the pathogens' viability. Similarly, the disinfectant eradicates most of the pathogens, while having a prolonged negative effect on the pathogens' infectivity.

The ionized and humid gases have a prolonged negative effect on the pathogens' infectivity. The ultraviolet radiation has a prolonged negative effect on the pathogens as well, by means of genetic destabilization. The added oxygen eases breathing. If necessary, the patient can further be treated with a nebulizer can be used to deliver bronchodilator (airway-opening) medications such as albuterol, Xopenex or Pulmicort (steroid). A nebulizer atomizes liquid (usually saline), often including medicine, into an inhalable gas form. By contrast, the instant device and method employs a dry gas (heated air) as the base material for the therapy. Oxygen (and/or other gases) may be added to the dry, heated air. Likewise, disinfectant may be added to the base heated air, alone or in conjunction with the added gas or gases.

### Process

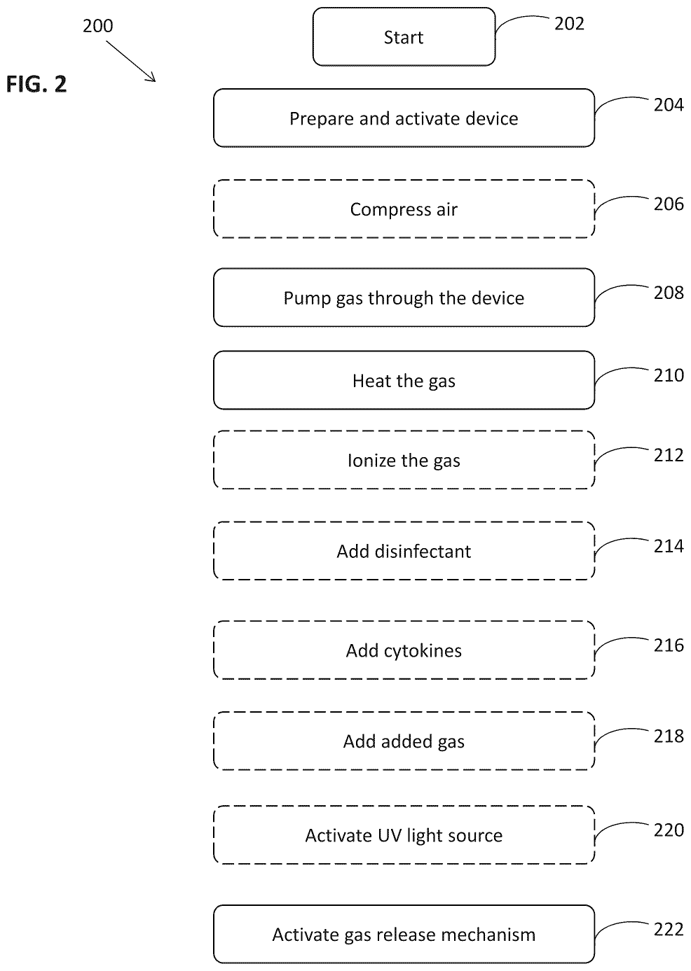

A process 200 for preparing and using device 10 for respiratory therapy is detailed hereafter with reference to FIG. 2. FIG. 2 is a flow diagram of a process 200 for preparing and using device 10 and/or 10′ for respiratory therapy. It is made clear that the steps detailed hereafter, unless initial or terminal steps, are not necessarily intended to be consecutive or sequential. That is to say that while the steps are described in logical order and according to the embodiments depicted in FIGS. 1 and 1A, this order is not intended to be limiting in any way.

Process 200 starts at block 202. At step 204, the air therapy device 10/10′ is prepared and activated. As part of the preparation of the device, if applicable (some embodiments of the device do not include a disinfectant container) and/or deemed necessary, reservoir 124 is filled with a measured amount of disinfectant.

If applicable (some embodiments of the device do not include an inlet port for an added gas) and/or deemed necessary, an external added gas source is mechanically coupled to inlet port 116, so as to be in fluid communication with the inlet port 116 and in controlled fluid communication with the gas in the internal volume of device 10. Preferably, said communication is controlled by at least one valve of the inlet port 116.

If applicable (some embodiments of the device do not include a cytokine compartment) and/or deemed necessary, compartment 130 is filled with a measured amount of cytokine fluid.

The temperature control dial A 174 is set to the prescribed temperature. Air pump 152 is activated, starting the process of providing heated dry gas (with or without various additives). Air pump 152 pumps air into device 10 from external, ambient air.

At optional (indicated by a block with a broken-line border) step 206, the gas pumped into the internal volume of the device is compressed by a compressor, such that the gas in the internal volume of device 10/10′ is under a predefined amount of pressure. According to this optional step, air pump 152 is a compressor or a compressor is provided in addition to air pump 152. The compressor compresses the ambient air into pressurized gas and forces this pressured air into the heating compartment and, once heated, into the holding area of fluid passage 111.

At step 208, dry gas (e.g. air) is pumped through the internal volume of the device 10, 10′. According to embodiments, the gas is pressurized prior to being expelled through the internal volume of the device. According to other embodiments, the gas is pressurized at a later stage. To clarify this latter point, in preferred embodiments, at least a portion of the internal volume of the device 10/10′ includes a pressurized section for holding the heated gas under pressure. For example, fluid passage 111, in preferred embodiments, stores the heated gas (heated air and/or added gas and/or disinfectant and/or cytokines) in a pressurized conduit.

At step 210, the gas in the internal volume of device 10 (whether pressurized or not) is heated by the heating component (e.g. heating element 142).

At optional step 212 (e.g. according to the embodiment of device 10′), gas in the internal volume of the device is ionized. As mentioned, the gas may be ionized at any stage prior to being expelled from the device.

At optional step 214, in embodiments including a disinfectant reservoir 124 (or similar component), if necessary, the disinfectant is released from the reservoir into the internal volume of the device to mix with the [pressurized] air.

At optional step 216, in embodiments including a cytokine compartment 130 (or similar component), if necessary, cytokine fluid is released from the compartment into the internal volume of the device to mix with the [pressurized] air.

At optional step 218, in embodiments including an inlet port for facilitating fluid communication of an added gas (e.g. oxygen), either ionized or not, or gas mixture, if necessary, the gas additive is introduced into the internal volume of the device to mix with the (preferably pressurized) air, via inlet port 116.

At optional step 220, in embodiments including an ultraviolet lamp 115 (or similar component), if necessary, ultraviolet radiation is beamed from the ultraviolet lamp 115 into the internal volume of the device to mix with the [pressurized] air. Introduction of disinfectant and/or additive gas and/or cytokine fluid may come before, after or during the heating stage of the process. The heated gas is pushed/pressured into the pressurized holding area, heretofore referred to as fluid passage 111. The heated gas, either ionized or not, is held in the fluid passage until released/presented to the user (via tubing and mask).

Before the heated gas is released, the speed of release is selected. There are at least two general options for releasing the heated gas: (1) relative slow release; or (2) relative fast/one-time release. Exemplarily, the release manner/speed is selected using dial B.

As detailed elsewhere, the fluid (liquid or gas) disinfectant and/or cytokines can be added prior to release of the heated gas from the device. Alternatively, the disinfectant and/or cytokines is/are released at the same time as the heated gas is released. In the latter case, the flow rate of the disinfectant fluid is selected and set, e.g. by dial C. Additionally or alternatively, the flow rate of the cytokine fluid is selected and set, e.g. by dial F.

Where applicable, the flow rate of the added (external) gas is set. Exemplarily, the added gas flow rate is selected and set using dial D 180. This option is relevant where the external gas has not been added to the native gas prior to release, and will only be added while the native, heated gas is being released. Disinfectant, cytokine and ultraviolet radiation may be added at the same time or prior to release.

In step 220 release mechanism is actuated (e.g. by pushing button F 184, or another user control, not shown) and the heated gas is released to the user. Tubing and a mask must be connected to the outlet port to be ready for presentation to the user. The user breathes the gas in using deep breaths (if possible). The release mechanism may further regulate the release of the heated gas, e.g. according to previously defined release rate settings.

In another possible configuration the same principles of device 10/10′ can be applied to an institutional-sized therapy compartment. According to the instant configuration, the compartment is proportioned such that the entire body of the patient can fit into the compartment. The temperature range of the heated gas is between 30° C. and 120° C.

The direct heat eradicates (usually at ˜50° C.) most of the pathogens, while having a prolonged negative effect on the pathogens' viability. Similarly, the disinfectant eradicates most of the pathogens, while having a prolonged negative effect on the pathogens' viability.

The ionized and humid gas has a prolonged negative effect on the pathogens' infectivity. The ultraviolet radiation has a prolonged negative effect on the pathogens as well. The added oxygen eases breathing. If necessary, the patient can further be treated with a nebulizer can be used to deliver bronchodilator (airway-opening) medications such as albuterol, Xopenex or Pulmicort (steroid).

A nebulizer atomizes liquid (usually saline), often including medicine, into an inhalable gas form. By contrast, the instant device and method employs a dry gas (heated air) as the base material for the therapy. Oxygen (and/or other gases) may be added to the dry, heated air. Likewise, disinfectant may be added to the base heated air, alone or in conjunction with the added gas or gases.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A device for providing an inhalable heated dry gas to a user, the device comprising:
an air pump adapted to draw ambient dry gas into the device and pump said gas through the device;
a heating component adapted to heat said dry gas pumped by said air pump, resulting in dry, heated gas; and
an outlet port through which said heated dry gas is expelled from the device.

2. The device of claim 1, wherein said air pump is an air compressor, wherein said pumped dry gas is compressed by said air compressor.

3. The device of claim 1, wherein said heating component is selected from the group consisting: a heating element, a laser, an infrared light.

4. The device of claim 1, further comprising an electrical source, said electrical source adapted to ionized said dry, heated gas.

5. The device of claim 1, wherein said dry gas is heated to a temperature in a range between 40° C. and 70° C.

6. The device of claim 1, wherein said dry gas is heated to a temperature in a range between 50° C. and 60° C.

7. The device of claim 1, wherein said dry gas is pumped through a pipe disposed within the device.

8. The device of claim 1, wherein said outlet port further includes an ultraviolet light source.

9. The device of claim 1, further comprising a gas inlet port adapted to communicate therethrough an added gas from an external source to intermix with said dry gas.

10. The device of claim 9, wherein said added gas is an ionized gas.

11. The device of claim 9, wherein said gas inlet port further includes a valve adapted to selectively control access of said added gas into the device so as to intermix with said dry gas.

12. The device of claim 1, further comprising a reservoir for holding a disinfectant additive to be intermixed with said dry gas in the device.

13. The device of claim 1, further comprising a cytokine compartment for holding a cytokine additive to be intermixed with the dry gas in the device.

14. A method of providing a heated dry gas, the method comprising:
pumping a dry gas into a device and propelling said dry gas through an internal volume of said device with an air pump of said device;
heating said dry gas inside said device by a heating component of said device so as to receive a heated dry gas; and
expelling said heated dry gas out of an outlet port of said device.

15. The method of claim 14, further comprising: compressing said dry gas with a compressor.

16. The method of claim 14, further comprising: ionizing said dry gas.

17. The method of claim 14, further comprising: adding a disinfectant fluid to intermix with said dry gas.

18. The method of claim 14, further comprising: adding a cytokine fluid to intermix with said dry gas.

19. The method of claim 14, further comprising: adding an added gas to intermix with said dry gas.

20. The method of claim 19, wherein said added gas is an ionized gas.

21. The method of claim 14, further comprising: radiating said dry gas with ultraviolet radiation.
